(19) United States
(12) Patent Application Publication
(10) Pub. No.: US 2017/0000377 A1
(43) Pub. Date: Jan. 5, 2017

(54) SYSTEM AND METHOD FOR IMAGING FREE RADICALS

(71) Applicant: THE GENERAL HOSPITAL CORPORATION, Boston, MA (US)

(72) Inventors: Matthew S. Rosen, Somerville, MA (US); Mathieu Sarracanie, Somerville, MA (US); Brandon Armstrong, Brighton, MA (US); Cenk Ayata, Sudbury, MA (US); Fanny Herisson, Camgridge, MA (US); Peter Caravan, Cambridge, MA (US); Eric Gale, Cambridge, MA (US)

(73) Assignee: THE GENERAL HOSPITAL CORPORATION, Boston, MA (US)

(21) Appl. No.: 15/125,676

(22) PCT Filed: Mar. 16, 2015
(86) PCT No.: PCT/US15/20738

§ 371 Date: Sep. 13, 2016

Related U.S. Application Data
(60) Provisional application No. 61/953,452, filed on Mar. 14, 2014.

(51) Int. Cl. A61B 5/055; G01R 33/60; G01R 33/28; A61B 5/00; G01R 33/385

(52) U.S. Cl. CPC A61B 5/055; A61B 5/7275; A61B 5/4064; A61B 5/4848; A61B 5/742; A61B 5/7475; A61B 5/0022; G01R 33/385; G01R 33/281; G01R 33/60

(57) ABSTRACT

A system and method performing a medical imaging process includes arranging a subject to receive an exogenously administered free radical probe, performing an Overhauser-enhanced MRI (OMRI) imaging process to acquire data from the subject, and reconstructing the data to generate a report indicating a spatial distribution of free radicals in the subject.

### CROSS-REFERENCE TO RELATED APPLICATIONS

This application is based on, claims priority to, and incorporates herein by reference, U.S. Provisional Application Ser. No. 61/953,452, filed Mar. 14, 2014, and entitled “SYSTEM AND METHOD FOR IMAGING FREE RADICALS USING MAGNETIC RESONANCE IMAGING SYSTEMS AND PROBES.”

### STATEMENT REGARDING FEDERALLY SPONSORED RESEARCH

This invention was made with government support under W81XWH-11-2-076 awarded by the Department of Defense. The government has certain rights in the invention.

### BACKGROUND

The present disclosure relates to systems and methods for the invention is magnetic resonance imaging (MRI). More particularly, the present disclosure provides systems and methods for imaging free radicals using MRI and probes.

When a substance such as human tissue is subjected to a uniform magnetic field (polarizing field B₀), the individual magnetic moments of the excited nuclei in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, M_{z}, may be rotated, or “tipped”, into the x-y plane to produce a net transverse magnetic moment Mₜ. A signal is emitted by the excited nuclei or “spins”, after the excitation signal B₁ is terminated, and this signal may be received and processed to form an image.

When utilizing these “MR” signals to produce images, magnetic field gradients (Gₓ, G_{y}, and G_{z}) are employed. Typically, the region to be imaged is scanned by a sequence of measurement cycles in which these gradients vary according to the particular localization method being used. The resulting set of received MR signals are digitized and processed to reconstruct the image using one of many well known reconstruction techniques.

Acute reperfusion therapies have changed ischemic stroke care, but treatments are limited because of a short therapeutic window owing to the risk of reperfusion injury and hemorrhage. Detection of early and mild blood-brain barrier (BBB) disruption is an unmet need in acute stroke diagnosis. Although contrast from relaxation-based MRI contrast agents such as Gd-DTPA is correlated with hemorrhagic transformation of an infarct, it is not sensitive enough to probe more mild BBB disruption.

Thus, further developments are necessary to meet clinical needs.

### SUMMARY

The present invention overcomes the aforementioned drawbacks by providing a system and method for Overhauser-enhanced MRI (OMRI) in conjunction with an injected stable free radical.

In accordance with one aspect of the disclosure, a magnetic resonance imaging (MRI) system is disclosed that is configured to perform an imaging process of a subject having received an exogenously administered free radical probe. The system includes a magnet system configured to generate a static magnetic field about at least a region of interest (ROI) of the subject arranged in the MRI system and at least one gradient coil configured to establish at least one magnetic gradient field with respect to the static magnetic field. The system also includes a radio frequency (RF) system configured to deliver excitation pulses to the subject and a computer system. The computer system is configured to control the at least one gradient coil and the RF system to perform a magnetic resonance (MR) imaging pulse sequence and, while performing the MR pulse sequence, perform electron spin resonance (ESR) pulses. The computer system is further configured to acquire data corresponding to signals from the subject having received an exogenously administered free radical probe excited by the MR pulse sequence and the ESR pulses and reconstruct, from the data, at least one anatomical image of the subject and spatially distributed free radicals within the subject relative to the anatomical image.

In accordance with another aspect of the disclosure, a method is provided for performing a medical imaging process. The method includes arranging a subject to receive an exogenously administered free radical probe and performing an Overhauser-enhanced magnetic resonance imaging (OMRI) process to acquire data from the subject. The method also includes reconstructing the data to generate a report indicating a spatial distribution of free radicals in the subject.

The foregoing and other advantages of the invention will appear from the following description.

### BRIEF DESCRIPTION OF THE DRAWINGS

FIG. 1 is a block diagram of an MRI system.

FIG. 2 is a block diagram of an RF system of an MRI system.

FIG. 3 is a picture of a low-field MRI (IfMRI) system in accordance with the present disclosure.

FIG. 4 is a picture of a probe for OMRI in accordance with the present disclosure.

FIG. 5 is a series of images including a photo of a TEMPOL concentration phantom and images thereof.

FIG. 6 is a set of images showing OMRI magnitude and phase images acquired from a rat at 6.5 mT following 75 min right MCAO and 60 min reperfusion. Four coronal slices from 10 slice data set shown. OMRI (NA=10) imaging time was 195 seconds. Low-resolution anatomical MRI (NA=80) was acquired in the OMRI scanner at 6.5 mT with DNP pulses disabled. MRI imaging time was 17 min. All images, voxel size: 1.1×1.6×8 mm3, TE/TR: 18/36 ms, Matrix: 128 x 35×10

### DETAILED DESCRIPTION

Overhauser-enhanced MRI (OMRI) is a promising technique for imaging free radicals, and a recently developed fast high-resolution OMRI methodology (Sarracanie M et al. MRM 2013 DOI: 10.1002/mrm.24705, which is incorporated herein by reference in its entirety) offers new perspectives for the imaging of free radicals in living organisms. As will be described, the present disclosure provides a method to probe hyperacute BBB breakdown following ischemic stroke using OMRI in conjunction with an injected stable free radical. However, before discussing these systems and methods, a general discussion of MR systems is provided.

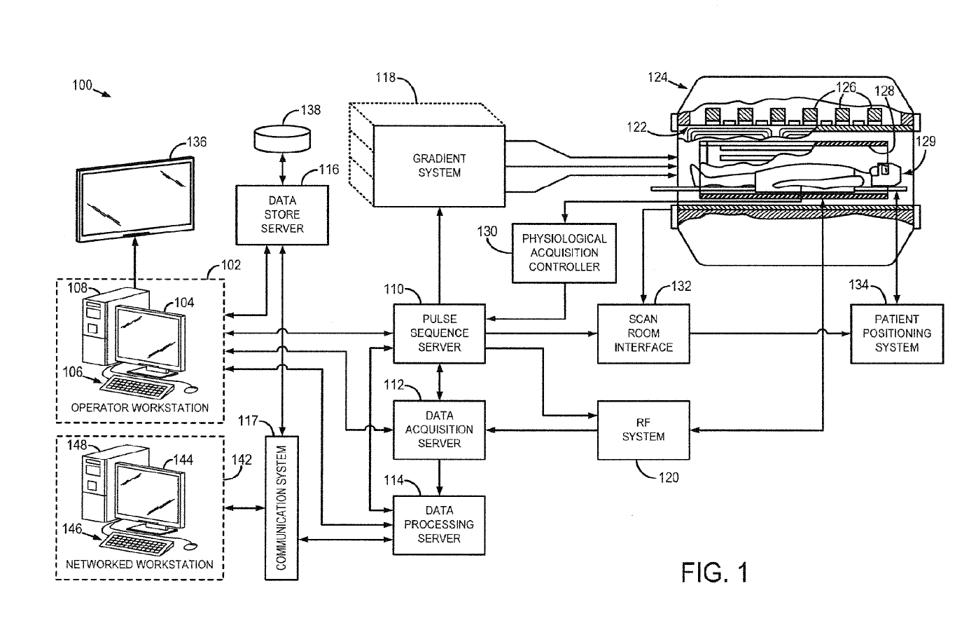

Referring particularly now to FIG. 1, an example of a magnetic resonance imaging (MRI) system 100 is illustrated. The MRI system 100 includes an operator workstation 102, which will typically include a display 104, one or more input devices 106, such as a keyboard and mouse, and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to four servers: a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 117, which may include any suitable network connection, whether wired, wireless, or a combination of both. As an example, the communication system 117 may include both proprietary or dedicated networks, as well as open networks, such as the internet.

The pulse sequence server 110 functions in response to instructions downloaded from the operator workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding magnetic resonance signals. The gradient coil assembly 122 forms part of a magnet assembly 124 that includes a polarizing magnet 126 and a whole-body RF coil 128 and/or local coil, such as a head coil 129.

RF waveforms are applied by the RF system 120 to the RF coil 128, or a separate local coil, such as the head coil 129, in order to perform the prescribed magnetic resonance pulse sequence. Responsive magnetic resonance signals detected by the RF coil 128, or a separate local coil, such as the head coil 129, are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays, such as the head coil 129.

The RF system 120 also includes one or more RF receiver channels. Each RF receiver channel includes an RF preamplifier that amplifies the magnetic resonance signal received by the coil 128/129 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (1)

and the phase of the received magnetic resonance signal may also be determined according to the following relationship:

$$ϕ = {tan}^{- 1} ( \frac{Q}{I} ) . \qquad ( 2 )$$

The pulse sequence server 110 also optionally receives patient data from a physiological acquisition controller 130. By way of example, the physiological acquisition controller 130 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data is lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data is acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also be employed to process magnetic resonance signals used to detect the arrival of a contrast agent in a magnetic resonance angiography (MRA) scan. By way of example, the data acquisition server 112 acquires magnetic resonance data and processes it in real-time to produce information that is used to control the scan.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 114 are conveyed back to the operator workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 102, or in a different facility, such as a different healthcare institution or clinic.

The networked workstation 142, whether within the same facility or in a different facility as the operator workstation 102, may gain remote access to the data processing server 114 or data store server 116 via the communication system 117. Accordingly, multiple networked workstations 142 may have access to the data processing server 114 and the data store server 116. In this manner, magnetic resonance data, reconstructed images, or other data may exchanged between the data processing server 114 or the data store server 116 and the networked workstations 142, such that the data or images may be remotely processed by a networked workstation 142. This data may be exchanged in any suitable format, such as in accordance with the transmission control protocol (TCP), the internet protocol (IP), or other known or suitable protocols.

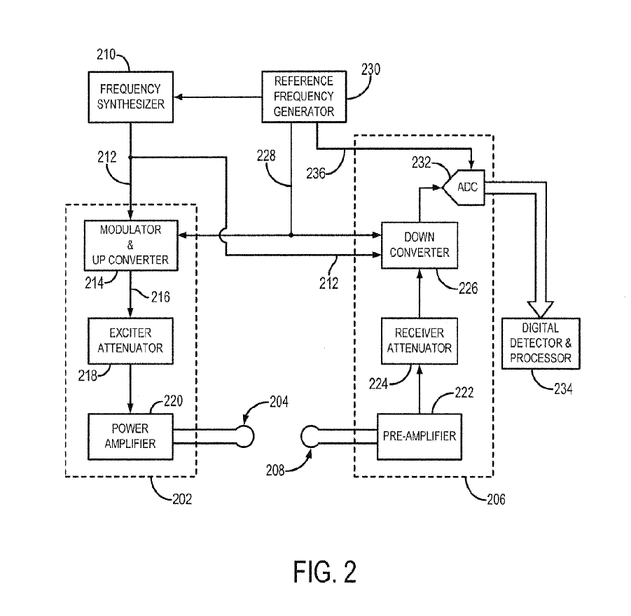

With reference to FIG. 2, the RF system 120 of FIG. 1 will be further described. The RF system 120 includes a transmission channel 202 that produces a prescribed RF excitation field. The base, or carrier, frequency of this RF excitation field is produced under control of a frequency synthesizer 210 that receives a set of digital signals from the pulse sequence server 110. These digital signals indicate the frequency and phase of the RF carrier signal produced at an output 212. The RF carrier is applied to a modulator and up converter 214 where its amplitude is modulated in response to a signal, R(t), also received from the pulse sequence server 110. The signal, R (t), defines the envelope of the RF excitation pulse to be produced and is produced by sequentially reading out a series of stored digital values. These stored digital values may be changed to enable any desired RF pulse envelope to be produced.

The magnitude of the RF excitation pulse produced at output 216 is attenuated by an exciter attenuator circuit 218 that receives a digital command from the pulse sequence server 110. The attenuated RF excitation pulses are then applied to a power amplifier 220 that drives the RF transmission coil 204.

The MR signal produced by the subject is picked up by the RF receiver coil 208 and applied through a preamplifier 222 to the input of a receiver attenuator 224. The receiver attenuator 224 further amplifies the signal by an amount determined by a digital attenuation signal received from the pulse sequence server 110. The received signal is at or around the Larmor frequency, and this high frequency signal is down converted in a two step process by a down converter 226. The down converter 226 first mixes the MR signal with the carrier signal on line 212 and then mixes the resulting difference signal with a reference signal on line 228 that is produced by a reference frequency generator 230. The down converted MR signal is applied to the input of an analog-to-digital (“ND”) converter 232 that samples and digitizes the analog signal. The sampled and digitized signal is then applied to a digital detector and signal processor 234 that produces 16-bit in-phase (I) values and 16-bit quadrature (Q) values corresponding to the received signal. The resulting stream of digitized I and Q values of the received signal are output to the data acquisition server 112. In addition to generating the reference signal on line 228, the reference frequency generator 230 also generates a sampling signal on line 236 that is applied to the A/D converter 232.

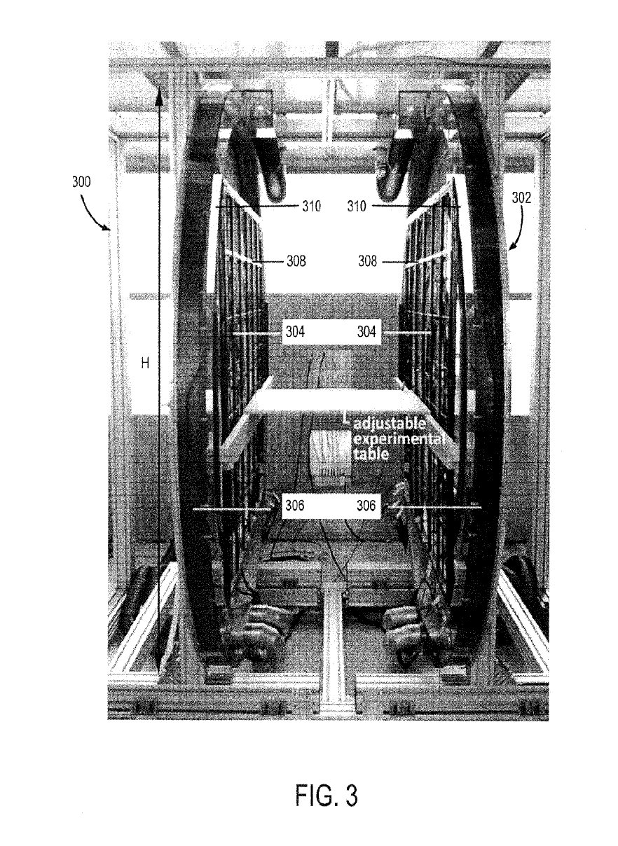

The basic MR systems and principles described above may be used to inform the design of other MR systems that share similar components but operate at very-different parameters. In one example, a low-field magnetic resonance imaging (IfMRI) system utilizes much of the above-described hardware, but has substantially reduced hardware requirements and a smaller hardware footprint. For example, referring to FIG. 3, a system 300 is illustrated that, instead of a 1.5 T or greater static magnetic field, utilizes a substantially smaller magnetic field. That is, in FIG. 3, as a non-limiting example, a 6.5 mT electromagnet-based scanner is illustrated. In particular, the system 300 includes a biplanar 6.5 mT electromagnet (B0) 302 that, for example, may be formed by inner B0 coils 304 and outer B0 coils 306. Biplanar gradients 308 may extend across the B0 electromagnet 302.

The system 300 may be tailored for ¹H imaging by achieving a high B0 stability, high gradient slew rates, and low overall noise. To achieve these ends, a power supply, for example, with +/−1 ppm stability over 20 min and +/−2 ppm stability over 8 h, may be used and high current shielded cables may be deployed throughout the system 300. In one non-limiting example, a power supply was adapted from a System 854T, produced by Danfysik, Taastrup, Denmark. The system 300 can operate inside a double-screened enclosure (ETS-Lindgren, St. Louis, Mo.) with a RF noise attenuation factor of 100 dB from 100 kHz to 1 GHz. In this example, the system may have a height, H, that is, as a non-limiting example, 220 cm. A cooling systems 310, such as may include air-cooling ducts, may be included.

Using the above-described system, TEMPOL (4-hydroxy-TEMPO) may be detected with very-high sensitivity by performing an OMRI process. TEMPOL, as used herein, refers to 4-Hydroxy-TEMPO 4-hydroxy-2,2,6,6-tetramethylpiperidin-1-oxyl. It is a heterocyclic compound. The present disclosure recognizes that, in a normal physiological state, TEMPOL does not cross the BBB. However, the present disclosure further recognizes that, because of its small size (172 Da), however, TEMPOL can cross the BBB under pathological circumstances associated with early BBB opening (e.g. ischemia), and act as an OMRI-detectable tracer. The use of TEMPOL as a small, exogenous OMRI agent allows monitoring BBB disruption in stroke at the hyperacute stage, much earlier than the traditional relaxation-based MRI contrast agents that rely on the leakage of larger molecules (such as Gd-DTPA) across the BBB.

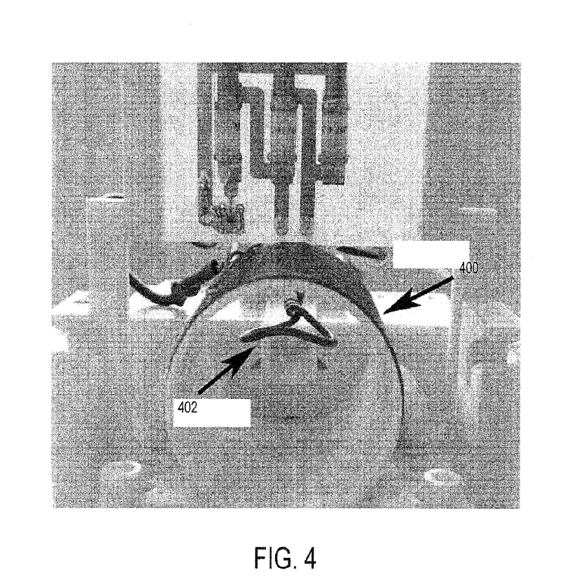

A 3D OMRI process can be performed using a variation of a balanced steady state free precession (b-SSFP) pulse sequence, for example, such as described in Sarracanie M et al. MRM 2013 DOI: 10.1002/mrm.24705. To achieve sensitivity of b-SSFP-based OMRI to free radical concentration, an NMR/ESR coil setup can be used. For one in vivo experiment, a single loop ESR coil 400 was arranged inside a solenoid NMR coil 402, as illustrate din FIG. 4. Under anesthesia, MCAO occlusion was performed in a 3 month old Wistar rat by insertion of filament via external carotid artery. Following 75 min MCAO and 60 min reperfusion, 3.6 μl/gbw of 300 μM TEMPOL was injected into the carotid artery after which the animal was sacrificed and OMRI imaging begun.

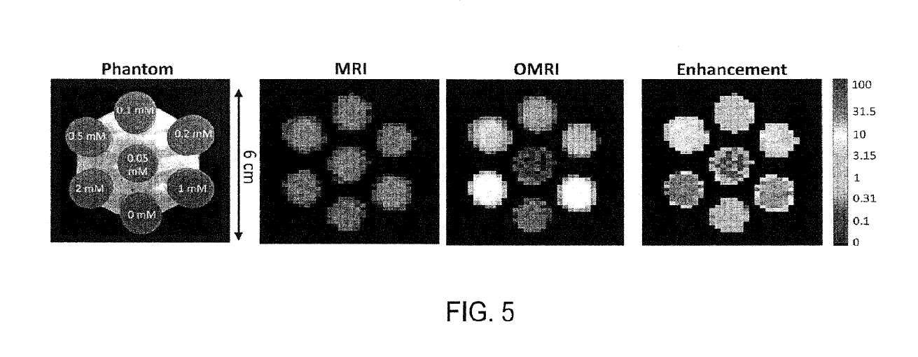

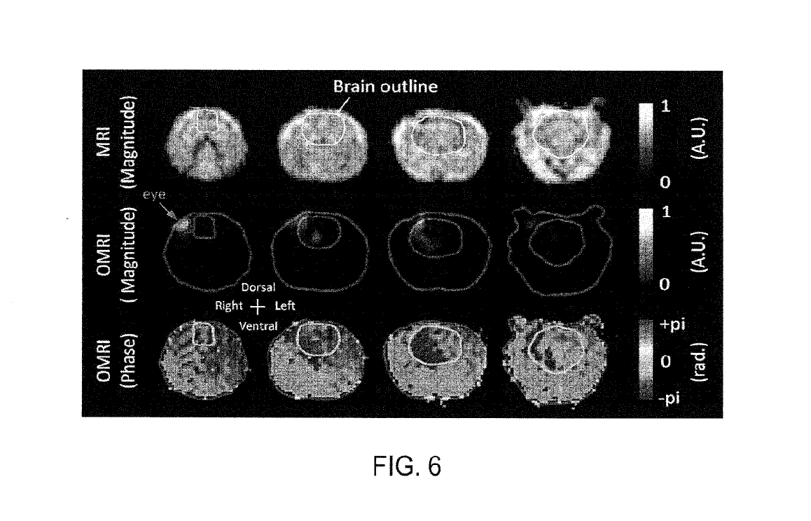

The sensitivity of b-SSFP-based OMRI to free radical concentration is shown in FIG. 5. The OMRI images demonstrate marked image-based free radical sensitivity. The OMRI enhancement image may be computed from a ratio of OMRI to MRI magnitude. In vivo OMRI signal enhancement in the frontal lobe and eye ipsilateral to the ischemic site is clearly visible in the OMRI images following reperfusion, as shown in FIG. 6. The phase of the OMRI image in FIG. 6 provides sensitive contrast even in cases where the radical concentration is very low and the Overhauser enhancement may be small.

Imaging has been performed using TEMPOL at low concentrations with OMRI methods in vitro, and crossing the BBB following ischemia/reperfusion in vivo. Thus, in accordance with the present disclosure, OMRI may be used in conjunction with stable free radical TEMPOL as an exogenously administered probe in hyperacute stroke. Also, in accordance with the present disclosure, TEMPOL may be used as a probe for observing early BBB breakdown following reperfusion. Additionally, as TEMPOL reduction has been used as a functional probe to study redox status in tissue, temporally resolved OMRI may be used to indicate the redox status of ischemic tissue.

Thus, a system and method is described for Overhauser-enhanced MRI (OMRI) that can be used in combination with an exogenously administered free radical probe to, for example, tomographically probe blood brain barrier breakdown and tissue oxidative stress status in vivo. Use of a small molecular size free radical probe molecule, for example, allows detection of hyper-acute and mild BBB disruption. Time-dependent tomographic measurements of free radical concentration reveal the extent of local redox status as a new functional probe. Exogeneously administered free radical probe molecule may be specifically functionalized to serve as molecular imaging target, binding to fibrin, for instance.

As opposed to relaxation-based MRI contrast mechanisms, free-radical OMRI signal enhancement can be modulated as desired by pulse sequence control of the ESR/electron paramagnetic resonance (EPR) drive field, enabling detection down to very-low radical concentrations to be made using a lock-in technique even in cases where the enhancement is small. The present disclosure advantageously provides a non-invasive, fast operation and reduced SAR at low magnetic field with b-SSFP OMRI sequences.

Free radical imaging, by providing a tomographic measurement of oxidative stress, has the potential to transform both the research and clinical management of stroke. A better understanding of stroke physiopathology and stroke models can be achieved by studying BBB disruption in animals in vivo. The present disclosure offers a new tool for drug design and neuroprotection studies.

Future clinical application of OMRI include acute stroke management (e.g., as a decision-making and prognostic tool), clinical research (including a more fundamental understanding of hemorrhagic transformation), and as a monitor of drug design and effectiveness. The ability to image and monitor very-early BBB disruption upon I/R, may also predict and prevent hemorrhagic transformation and complications of stroke treatments such as thrombolysis.

The use of OMRI to image free radicals in vivo is an advantageous tool applicable to other neurologic diseases in which oxidative stress appears to play a significant role such as head trauma, Alzheimer's dementia and multiple sclerosis, and even in other organ systems. In cancer, measurement of tumor redox state in response to therapy may aid development of chemoprevention strategies, as well as monitor the impact of therapies directed to alleviate free radical-mediated cell damage.

The present invention has been described in terms of one or more embodiments, and it should be appreciated that many equivalents, alternatives, variations, and modifications, aside from those expressly stated, are possible and within the scope of the invention.

1. A magnetic resonance imaging (MRI) system configured to perform an imaging process of a subject having received an exogenously administered free radical probe, comprising:
a magnet system configured to generate a static magnetic field about at least a region of interest (ROI) of the subject arranged in the MRI system;
at least one gradient coil configured to establish at least one magnetic gradient field with respect to the static magnetic field;
a radio frequency (RF) system configured to deliver excitation pulses to the subject;
a computer system programmed to:
control the at least one gradient coil and the RF system to perform a magnetic resonance (MR) imaging pulse sequence;
while performing the MR pulse sequence, perform electron spin resonance (ESR) pulses;
acquire data corresponding to signals from the subject having received an exogenously administered free radical probe excited by the MR pulse sequence and the ESR pulses; and
reconstruct, from the data, at least one anatomical image of the subject and spatially distributed free radicals within the subject relative to the anatomical image.

2. The system of claim 1 further comprising an electron paramagnetic resonance (EPR) drive and wherein the computer system is further programmed to modulate the signal controlling the EPR drive using the MR pulse sequence.

3. The system of claim 1 wherein the static magnetic field includes a low-field static magnetic field.

4. The system of claim 3 wherein the static magnetic field is less than 10 mT

5. The system of claim 1 wherein the computer system is further programmed to control of the ESR pulses and drive field using the MR pulse sequence.

6. The system of claim 1 wherein the at least one anatomical image indicates a ratio of OMRI signals to MRI signal magnitudes.

7. The system of claim 1 wherein the report includes time-dependent tomographic measurements of free radical concentration relative to spatial locations in the subject.

8. The system of claim 1 wherein the exogenously administered free radical probe includes 4-hydroxy-TEMPO.

9. A method for performing a medical imaging process, the method comprising:
arranging a subject to receive an exogenously administered free radical probe;
performing an Overhauser-enhanced magnetic resonance imaging (OMRI) process to acquire data from the subject; and
reconstructing the data to generate a report indicating a spatial distribution of free radicals in the subject.

10. The method of claim 9 wherein the free radical probe is configured to probe at least one of blood brain barrier (BBB) breakdown and tissue oxidative stress status in the subject.

11. The method of claim 9 wherein the free radical probe is functionalized or targeted to bind to a particular organ or a tissue of interest.

12. The method of claim 11 wherein the target binds to at least one of fibrin, collagen, arterial or venous plaques, or tumor cells.

13. The method of claim 9 wherein the report indicates at least one of hyper-acute or mild BBB disruption.

14. The method of claim 9 further comprising developing a chemoprevention strategy using the report.

15. The method of claim 9 further comprising using the report to predict or prevent hemorrhagic transformation.

16. The method of claim 9 further comprising delivering therapies directed to alleviate free radical-mediated cell damage and monitoring an impact of the therapies using the report.

17. The method of claim 16 wherein the cell damage is caused by at least one of TBI, stroke, cancer, and multiple sclerosis.

18. The method of claim 9 wherein the report includes time-dependent tomographic measurements of free radical concentration relative to spatial locations in the subject.

19. The method of claim 9 wherein the exogenously administered free radical probe includes 4-hydroxy-TEMPO.

20. The method of claim 9 wherein the OMRI process includes using an electron spin resonance (ESR) drive field and wherein detection of free radical concentrations is made using a lock-in technique and synchronous ESR drive field.
